(19) United States
(12) Patent Application Publication
(10) Pub. No.: US 2024/0002941 A1
(43) Pub. Date: Jan. 4, 2024

(54) METHOD FOR PREDICTING PATIENT RESPONSE TO CD40-TARGETED THERAPIES

(71) Applicants: Albert Einstein College of Medicine, Bronx, NY (US); Montefiore Medical Center, Bronx, NY (US); Johannes Gutenberg University (JGU) Medical Center, Mainz (DE)

(72) Inventors: Yaron Tomer, Chappaqua, NY (US); Larissa C. Faustino, Bronx, NY (US); George J. Kahaly, Mainz (DE)

(21) Appl. No.: 18/254,242

(22) PCT Filed: Dec. 2, 2021
(86) PCT No.: PCT/US2021/061596

§ 371 Date: May 24, 2023

Related U.S. Application Data
(60) Provisional application No. 63/120,514, filed on Dec. 2, 2020.

(51) Int. Cl. C12Q 1/6883; C07K 16/28; G01N 33/68

(52) U.S. Cl. CPC C12Q 1/6883; C07K 16/2878; G01N 33/6893; C12Q 2600/106; G01N 2800/52; C12Q 2600/158; G01N 2333/70578; G01N 2800/24; C12Q 2600/156

(57) ABSTRACT

Methods of predicting response of a subject in need of immunosuppressant therapy to a Cluster of differentiation 40 (CD40)-targeted treatment are disclosed. The methods permit treating the subject with a treatment most likely to show a favorable response.

### CROSS-REFERENCE TO RELATED APPLICATION

This application claims priority of U.S. Provisional Application No. 63/120,514 filed Dec. 2, 2020, which is hereby incorporated by reference in its entirety.

### BACKGROUND

This disclosure is directed to methods of predicting patient response to CD40-targeted therapeutics and methods of treating a patient in need of immunosuppressant therapy.

Cluster of differentiation 40 (CD40) is an important co-stimulatory protein expressed on antigen presenting cells such as dendritic cells and B-cells. It is a member of the TNF receptor family and has many functions including: activation of B-cell proliferation, immunoglobulin class switching, antibody secretion, and the generation of long-lived memory cells. In addition, CD40 activation induces secretion of cytokines, such as IL-6, IL-8, and BAFF. CD40 is activated by CD40 ligand (CD40L). The interaction between CD40 and its ligand CD40L (also referred to as CD154) is critical for mounting an effective immune response against exogenous pathogens and naturally arising tumors. Consequently, breakdown in the homeostasis of the CD40/CD40L axis leads to both immunodeficiency and autoimmunity. Autoimmune diseases in which CD40 plays a pathogenic role include autoimmune thyroiditis, type 1 diabetes, inflammatory bowel disease, psoriasis, multiple sclerosis, rheumatoid arthritis, systemic lupus erythematosus, Graves' disease, and many others.

Almost 20 years ago, we discovered a SNP in the CD40 gene (rs1883832) that was strongly associated with Graves' disease (GD) and antibody mediated autoimmune thyroid disease (Tomer et al. Thyroid 2002; 12: 1129-1135). We also defined the mechanisms by which this CD40 SNP help trigger GD. The presence of the C-allele of rs1883832 (the “risk allele” or “susceptible allele”) drives increased CD40 expression leading to cytokine secretion (mainly IL-6). In contrast, the presence of the T-allele (the “protective allele”) correlates with decreased CD40 expression. Increased CD40 expression in the thyroid, such as that driven by the susceptible allele, triggered a strong cytokine response that could trigger thyroid autoimmunity and Graves' disease (Jacobson et al. Endocrinology 2005; 146: 2684-2691, Huber et al. J Immunol 2012: 189: 3043-3053, Lee et al. Endocrinology 2017; 158: 410-418). Subsequently, other CD40 SNPs were found to be associated with other autoimmune diseases such as Multiple Sclerosis (MS) and Rheumatoid Arthritis (RA).

As the importance of the CD40 gene in the pathoetiology of autoimmune diseases became clear, interest grew in targeting CD40 as a treatment of various autoimmune diseases, including Graves' disease. Novartis has an antagonistic anti-CD40 monoclonal antibody (mAb) called iscalimab (CFZ533) in development that has so far been shown to have efficacy in treating GD, RA, Myasthenia Gravis, Sjogren's syndrome, and in prevention of graft rejection after renal transplantation. Antagonistic anti-CD40 mAbs block CD40/CD40L interaction to abrogate downstream signaling and suppress unwanted immune responses. To date, at least five antagonistic anti-CD40 mAbs have entered clinical trials for various autoimmune diseases, including Graves' hyperthyroidism (Kahaly et al., OR19-6 A Novel Anti-CD40 Monoclonal Antibody, Iscalimab, Successfully Treats Graves' Hyperthyroidism, Journal of the Endocrine Society, Volume 3, Issue Supplement_1, April-May 2019, Journal of the Endocrine Society, Volume 3, Issue Supplement_1, April-May 2019, OR19-6, https://doi.org/10.1210/js.2019-OR19-6), primary Sjogren's syndrome (Fisher et al., 2017, Arthritis Rheumatol. 69 (Suppl. 10), 1784), rheumatoid arthritis (Visvanathan et al., 2016, Treatment with BI 655064 (antagonistic anti-CD40 antibody) modulates clinical and biomarker parameters associated with rheumatoid arthritis (RA) [abstract]. Arthritis Rheumatol. 68 (Suppl. 10), 1588.), plaque psoriasis (Anil Kumar et al., 2018, Randomized, controlled study of bleselumab (ASKP1240) pharmacokinetics and safety in patients with moderate-to-severe plaque psoriasis. Biopharm. Drug Dispos. 39, 245-255), Crohn disease (Kasran et al., 2005, Safety and tolerability of antagonist anti-human CD40 Mab ch5D12 in patients with moderate to severe Crohn's disease. Aliment. Pharmacol. Ther. 22, 111-122.), and ulcerative colitis (NCT03695185), as well as for transplant rejection (Farkash et al., 2019, Am. J. Transplant. 19 (Suppl. 3), 632).

However, one of the main challenges in using CD40-targeted therapies, such as the antibody iscalimab, is that not all patients show clinical improvement from treatment with a CD40-targeting therapeutic (“responders”). For example, a recently published paper reported results of a small study of iscalimab treatment in Graves' disease. Although 15 patients were treated with iscalimab, only 7 patients responded to the treatment (Kahaly et al. JCEM 2020; 105: 1-9).

Antibody therapeutics such as the CD40-targeting iscalimab are typically costly and can produce side effects or adverse effects. Currently, there is no way to predict which patients will respond favorably to treatment with a CD40-targeted therapy and which patients will not. It would be desirable to be able to predict if a given patient is likely to respond favorably to a CD40-targeted therapy, such as anti-CD40 antibodies, in order to minimize administration of costly treatments to patients unlikely to receive therapeutic benefit from the treatment. Thus, there is a need for methods predicting patient response to CD40-targeted therapies.

### SUMMARY

Disclosed are methods of predicting response of a subject with an autoimmune disease to a Cluster of differentiation 40 (CD40)-targeted treatment.

In one aspect, the method for predicting response of a subject to a Cluster of differentiation 40 (CD40)-targeted treatment includes genotyping Cluster of differentiation 40 (CD40) single nucleotide polymorphism (SNP) rs1883832 and/or rs4810485 in a biological sample from a subject in need of immunosuppressant therapy; and predicting that the subject will respond to treatment with a CD40-targeted active agent when the determined genotype of rs1883832 is homozygous for C or when the determined genotype of rs4810485 is homozygous for G, or predicting that the subject will not respond to treatment with a CD40-targeted active agent when the determined genotype of rs1883832 is heterozygous or homozygous for T or when the determined genotype of rs4810485 is heterozygous or homozygous for T.

In another aspect, the method for predicting response of a subject to a Cluster of differentiation 40 (CD40)-targeted treatment includes determining Cluster of differentiation 40 (CD40) expression levels in a biological sample from a subject in need of immunosuppressant therapy; and predicting that the subject will respond to treatment with a CD40-targeted active agent when the determined CD40 expression level is greater than 0.3% of GAPDH expression level in the biological sample, or predicting that the subject is unlikely to respond to treatment with a CD40-targeted active agent when the determined CD40 expression level is below 0.3% of GAPDH expression level in the biological sample.

These and other features and characteristics are more particularly described below.

### BRIEF DESCRIPTION OF THE DRAWINGS

The following is a brief description of the drawing, which is presented for the purposes of illustrating the exemplary embodiments disclosed herein and not for the purposes of limiting the same.

FIG. 1 is a graph showing expression of CD40 relative to GAPDH in patients with Haplotype Pair A (Non-Responders) compared to patients with Haplotype Pair B or C (Responders).

### DETAILED DESCRIPTION

The inventors have identified biomarkers in the human CD40 gene that permit prediction of patient response to treatment with therapeutics, such as monoclonal antibodies, that target CD40. The biomarkers are single nucleotide polymorphisms (SNPs) in the CD40 gene or expression level of the CD40 gene. A reference nucleotide sequence for the CD40 gene is GENBANK Accession No. NC_000020 Region: 46116499 to 46131599, Version No. NC_000020.11 (SEQ ID NO. 12).

The methods disclosed herein permit identification of a patient as a responder or a non-responder to the therapeutic targeting CD40. Methods of treating a patient, based on identification of the patient as a responder or non-responder, are also disclosed and permit physicians to administer CD40-targeted therapies only to the subset of patients that will likely benefit from such therapies and to administer other therapies to patients that will likely not benefit from CD40-targeted therapies. The predictive methods and the treatment methods are both advantageously applied to patients in need of immunosuppressant therapy, for example patients with autoimmune disorders or transplant candidates or recipients. Autoimmune diseases that may potentially be treated by CD40-targeted therapies affect up to 5% of the population.

Methods for predicting response of a subject to a Cluster of differentiation 40 (CD40)-targeted treatment are disclosed.

The term “subject” or “patient” refers to a living mammalian organism, such as a human, monkey, cow, sheep, goat, dog, cat, mouse, rat, guinea pig, or transgenic species thereof. In certain embodiments, the patient or subject is a primate. A preferred patient or subject is a human Non-limiting examples of human subjects are adults, juveniles, infants and fetuses.

“CD40-targeted treatment” refers to medical treatment of a symptom, disorder, or condition by administration of a CD40-targeted active agent. Preferably, the CD40-targeted active agent at least partially blocks interaction between CD40 and CD40L.

“Treatment” refers to any medical care given to a patient for a disorder or injury and can include administration of an active agent, a surgical procedure, or a behavioral therapy such as altering diet, exercise, stress levels, and the like.

An “active agent” is any compound, element, or mixture that when administered to a patient alone or in combination with another agent confers, directly or indirectly, a physiological effect on the patient. When the active agent is a compound, salts, solvates (including hydrates) of the free compound or salt, crystalline and non-crystalline forms, as well as various polymorphs of the compound are included. The compound can be a macromolecule such as a protein or nucleic acid.

The CD40-targeted active agent can be an antagonistic anti-CD40 antibody, or any other compound blocking CD40, preferably, a monoclonal antibody (mAb). Preferably the anti-CD40 antibody is a fully human or a humanized antibody. Antagonistic anti-CD40 mAbs block CD40/CD40L interaction to abrogate downstream signaling and suppress unwanted immune responses. Exemplary antagonistic anti-CD40 monoclonal antibodies include BI 655064, ch5D12, bleselumab (ASKP 1240), Abbv-323 (ravagalimab), and iscalimab (CFZ533).

“Responding” to a treatment refers to occurrence of a favorable post-treatment therapeutic effect in the subject's disorder or disease, such as inhibiting the disorder or disease, e.g., arresting the development of the disorder or disease, relieving the disorder or disease, causing regression of the disorder or disease, relieving a condition caused by the disease or disorder, or reducing the symptoms of the disease or disorder.

In one aspect, the method of predicting response of a subject to CD40-targeted treatment can comprise genotyping CD40 single nucleotide polymorphism (SNP) rs1883832 and/or rs4810485 in a biological sample from a subject in need of immunosuppressant therapy; and predicting that the subject will respond to treatment with a CD40-targeted active agent when the determined genotype of rs1883832 is homozygous for C or when the determined genotype of rs4810485 is homozygous for G, or predicting that the subject will not respond to treatment with a CD40-targeted active agent when the determined genotype of rs1883832 is heterozygous or homozygous for T or when the determined genotype of rs4810485 is heterozygous or homozygous for T.

“Immunosuppressant therapy” means a treatment that lowers the activity of the subject's immune system. Examples of a subject in need of immunosuppressant therapy include a patient with an autoimmune disease, a candidate for a transplant, and a recipient of a transplant.

The autoimmune disease can be any autoimmune disease thought to be treatable by targeting CD40 and blocking interaction with CD40L. Examples of such an autoimmune disease include Graves' disease, antibody mediated autoimmune thyroid disease, Rheumatoid arthritis (RA), Multiple Sclerosis (MS), Myasthenia Gravis, Sjogren's syndrome, Systemic Lupus Erythematosus, Crohn's disease, and a combination of the foregoing. Preferably, the autoimmune disease is Graves' disease. The American Autoimmune Related Diseases Association maintains a list of autoimmune diseases, available on its website, which includes additional autoimmune diseases thought to be treatable by targeting CD40 and blocking interaction with CD40L.

The transplant can be a tissue transplant or an organ transplant. Exemplary tissues include cornea, bone marrow, hematopoietic stem cells, and skin. Exemplary organs include a kidney, a liver, a heart, and a lung.

A transplant candidate means a subject who needs a new tissue or organ transplant.

A transplant recipient means a subject who has received a tissue or an organ transplant.

A “single nucleotide polymorphism” or “SNP” refers to a genetic variation in a single nucleotide, such as a replacement of cytosine (C) or guanine (G) with thymine (T) or adenine (A), at a specific location in the genome. SNPs are identified herein using rs identifier numbers in accordance with the National Center for Biotechnology Information (NCBI) Single Nucleotide Polymorphism database (dbSNP), a public-domain archive for a broad collection of simple genetic polymorphisms, available via the internet. As used herein, rs numbers refer to the dbSNP Homo sapiens build 154 released Apr. 21, 2020.

Herein, “biological sample” means any biological material from which nucleic acid molecules, preferably genomic DNA or total RNA, can be prepared. Non-limiting examples of suitable biological samples useful herein include whole blood, plasma, saliva, buccal swab, and other bodily fluids or tissues that contain nucleic acids. One preferred biological sample is whole blood.

As used herein, “nucleic acid” means a polynucleotide such as a single or double-stranded DNA or RNA molecule, including, for example, genomic DNA, cDNA, and mRNA. The term nucleic acid includes nucleic acid molecules of both natural and synthetic origin, as well as molecules of linear, circular, or branched configuration representing either sense or antisense strands, or both, of a native nucleic acid molecule.

The method can further comprise obtaining the biological sample from the subject. Any suitable method to obtain the biological sample can be used. For example, whole blood can be drawn from the subject or the subject can spit saliva into a collection vessel.

The method can further comprise administering a CD40-targeted active agent to the subject when the determined genotype of rs1883832 is homozygous for C or when the determined genotype of rs4810485 is homozygous for G; or administering a treatment that does not target CD40 to the subject when the determined genotype of rs1883832 is heterozygous or homozygous for T or when the determined genotype of rs4810485 is heterozygous or homozygous for T.

Herein, a “treatment that does not target CD40” refers to a treatment other than treatment with an active agent blocking interaction with CD40L, such as an antagonistic anti-CD40 antibody. The treatment that does not target CD40 can be any suitable immunosuppressant treatment known in the art, and can be, for example, administration of one or more active agents in which the mode of action does not directly target CD40, surgery, or a behavioral therapy involving alterations in diet, exercise, and/or stress levels. A suitable treatment regimen for a given autoimmune condition can be determined based on the latest guidelines for treating the autoimmune condition from expert medical societies. As an example, the most recently published Graves' Disease Guidelines developed by the American Thyroid Association® (ATA) and American Association of Clinical Endocrinologists or the most recent version of the European Thyroid Association Guideline for the Management of Graves' Hyperthyroidism (Eur Thyroid J 2018; 7:167-186) can be used to determine evidence-based recommendations for suitable treatment for Graves' Disease. For Graves' Disease non-CD40-targeted therapies can include radioactive iodine therapy; an antithyroid medication that interferes with the thyroid's use of iodine to produce hormones, such as propylthiouracil or methimazole (TAPAZOLE); a beta blocker, e.g. propranolol (Inderal, INNOPRAN XL), Atenolol (TENORMIN), Metoprolol (LOPRESSOR, TOPROL-XL), or Nadolol (CORGARD).

Immunosuppressant therapies for transplant recipients are known in the art. See for example, Hartono, C, et al Immunosuppressive Drug Therapy, Cold Spring Harb Perspect Med. 2013 September; 3(9): a015487. Examples of non-CD40 targeted immunosuppressant active agents include calcineurin inhibitors such as tacrilimus and cyclosporine; antiproliferative agents such as mycophenolate mofetil, mycophenolate sodium and azathioprine; mTOR inhibitors such as sirolimus and everolimus; corticosteroids such as prednisone; and biologics such as atgam, OKT3, thymoglobulin, basiliximab, daclizumab, adalimumab (HUMIRA), and rituximab (RITUXAN). Suitable treatment regimens can be identified in guidelines published by professional societies such as the American Society of Transplantation, KDIGO (Kidney Disease Improving Global Outcomes), and American Association for the Study of Liver Diseases.

The method can further comprise genotyping CD40 rs6074022; rs745307; rs11569309; rs3765457; rs112809897; a SNP within 1 million base pairs distance upstream or 1 million base pairs distance downstream from rs1883832 in strong linkage disequilibrium with rs1883832, wherein strong linkage disequilibrium is defined as coefficient of correlation (r) square (r2) value >0.7; or a combination thereof in the biological sample. The method can comprise genotyping each of rs1883832, rs6074022; rs745307; rs4810485; rs11569309; rs3765457; and rs112809897.

The method can further comprise determining a haplotype pair from the genotypes; and predicting that the subject will respond to a CD40-targeted treatment when the determined haplotype pair comprises a rs1883832 genotype homozygous for C and/or a rs4810485 homozygous for G, or predicting that the subject will not respond to a CD40-targeted treatment when the determined haplotype comprises a rs1883832 genotype heterozygous or homozygous for T and/or a rs4810485 genotype heterozygous or homozygous for T.

A “genotype” of a SNP refers to the allele(s) of the SNP present on one or both chromosomes of a subject, preferable the genotype refers to the alleles of the SNP present on both chromosomes of a subject.

A “haplotype” refers to a 5′ to 3′ sequence of alleles found at a set of one or more polymorphic sites in a locus, such as the CD40 gene, on a single chromosome of a subject.

A “haplotype pair” refers to the two haplotypes found for a locus in a subject.

The genotype of a SNP can be determined in a biological sample, by any suitable method. Many methods are available for detection of one or more alleles of a SNP, including sequencing methods, re-sequencing methods, amplification methods, and hybridization methods. Analysis of nucleic acids in a biological sample from an individual, whether amplified or not, may be performed using any of these methods. Exemplary methods include but are not limited to polymerase chain reaction (PCR), restriction fragment length polymorphism analysis (RFLP), reverse-transcription PCR (RT-PCR), isothermal amplification, 5′ fluorescence nuclease assay (e.g. TAQMAN assay), molecular beacon assays, heteroduplex mobility assays (HMA), single strand conformational polymorphism (SSCP), denaturing gradient gel electrophoresis (DGGE). planar microarrays, bead arrays, sequencing, chemical cleavage of mismatch (CCM), and denaturing high performance liquid chromatography (DHPLC). One of ordinary skill in the art would understand that any known method of amplification of a nucleotide could be incorporated into a method to detect one or more alleles of a SNP. One of ordinary skill in the art would further understand that these methods of amplification of a nucleotide could use DNA, RNA, or a combination of the two.

These assays may be multiplexed, meaning two or more reactions may be conducted simultaneously in the same physical location, such as in the same tube or on the same substrate, such as a biochip, ensuring that the reaction products of the multiplexed reactions can be distinguished. For example, TAQMAN or molecular beacon assays can be multiplexed by use of any by monitoring of accumulation or depletion of two different fluorochromes corresponding to different sequence specific probes.

As used herein, “PCR” is any method involving the amplification of a nucleotide sequence based upon complementary primer binding to a target sequence. One of ordinary skill in the art will understand that PCR may be employed as part of many techniques for identifying a SNP risk variant, including but not limited to Tetra-primer amplification refractory mutation system PCR (ARMS-PCR). In ARMS-PCR, primers are employed whose 3′ ends encompass the SNP location, with each primer encoding a different allele at the SNP location. The primers are also designed to produce different length amplification fragments, thus allowing discrimination of the SNP genotype based upon the length of the amplified fragments.

As used herein, “RFLP” is any method for distinguishing genetic polymorphisms using a restriction enzyme, which is an endonuclease that catalyzes the degradation of nucleic acid and recognizes a specific base sequence, generally a palindrome or inverted repeat. One of ordinary skill in the art would understand that the use of RFLP analysis depends upon an enzyme that can differentiate two alleles at a polymorphic site.

As used herein, “RT-PCR” is any method involving the amplification of a RNA sequence using a reverse transcriptase to produce a cDNA sequence, followed by amplification of a nucleotide sequence based upon complementary primer binding to a target sequence. One of ordinary skill in the art will understand that RT-PCR may be employed as part of many techniques for identifying a SNP risk variant.

As used herein, “isothermal amplification” is any method involving amplification of a nucleotide sequence based upon complementary primer binding to a target sequence performed at a constant temperature. One example of an isothermal amplification method is loop-mediated isothermal amplification (LAMP). Generally, LAMP is used to amplify from a DNA sequence and is performed using multiple primer sets and a polymerase with a high strand displacement activity. Another example of an isothermal amplification method is nucleic acid sequence based amplification (NASBA). Generally, NASBA is used to amplify from a RNA sequence and is performed using a reverse transcriptase, an RNAse, and a RNA polymerase.

As used herein, a “5′ fluorescence nuclease assay” is any method using a target allele specific probe bearing a 5′ fluorescent dye label. In general, when the allele specific probe is used to amplify the target sequence, the 5′-nuclease activity of the polymerase cleaves the 5′ fluorescent dye label off of the probe, changing the molecular weight of the fluorescent dye molecule and therefore changing the fluorescence polarization. This change in fluorescence polarization may be detected, thereby confirming the presence of the target allele.

As used herein, “hybridization methods” mean methods relying on the use of a labeled oligonucleotide probe having a sequence complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used to selectively amplify, for example, a non-disease-predisposing allele by using an allele-specific oligonucleotide primer that is complementary to the nucleotide sequence of the non-disease-predisposing allele but which has one or more mismatches as compared to other alleles. One of ordinary skill in the art will understand that the one or more nucleotide mismatches that distinguish between the disease-predisposing allele (or the non-disease promoting allele) and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer. Non-limiting examples of hybridization methods useful herein include molecular beacon assays.

As used herein, a “HMA assay” is useful for detecting the presence of a polymorphic sequence since a DNA duplex carrying a mismatch has reduced mobility in a polyacrylamide gel compared to the mobility of a perfectly base-paired duplex.

As used herein, “SSCP” can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

As used herein, “DGGE” can be used to detect SNPs by electrophoresis of double-stranded DNA in a gel containing an increasing concentration of denaturant. The double-stranded DNA fragments containing mismatched alleles will have segments that will likely melt more rapidly, causing such fragments to migrate at a different rate compared to perfectly complementary sequences.

When implementing methods for detection of one or more SNPs, an array may be used to perform a high-throughput assay. The array generally comprises one or more reagents, such as nucleic acid primers and/or probes, for identifying in a nucleic acid sample from a subject the occurrence of an allelic variation corresponding to one or more SNPs. These reagents may be immobilized onto a substrate in a spatially addressable manner, such that each reagent is located at a different, identifiable, position on the array. The substrate may include multi-welled plates, ceramic chips, or beads. In a non-limiting example, the substrate may be a 96 well dish, with each well constituting a reaction chamber within which separate reactions comprising identified constituents may be performed. The reaction constituents may include primers for amplifying DNA or probes for binding specific sequences and reaction reagents. The reagents may be in any suitable form, including in solution, dried, lyophilized, or glassified. In a further non-limiting example, the array may include two or more sets of beads, with each bead having an identifiable marker, such as a quantum dot or fluorescent tag, so that the beads may be individually identified using, for example, a flow cytometer. Various array technologies are commercially available, for example from Applied Biosystems. Informatics and/or statistical software or other computer-implemented processes for analyzing array data and/or identifying genetic risk factors from data obtained from a patient sample are well known in the art and would be readily understood by the ordinarily skilled artisan.

Other molecular methods useful for determining genotype of a SNP known in the art may also be used when performing the methods disclosed herein.

In some embodiments, SNPs in linkage disequilibrium with the SNPs shown to be predictive of response to CD40 targeted treatments are useful for obtaining similar results. As used herein, linkage disequilibrium refers to the non-random association of SNPs at two or more loci. Techniques for the measurement of linkage disequilibrium are known in the art. As two SNPs are in linkage disequilibrium if they are inherited together, the information they provide is correlated to a certain extent. SNPs in linkage disequilibrium with the SNPs included in the disclosed methods can be obtained from databases such as HapMap or other related databases, from experimental setups run in laboratories or from computer-aided in silico experiments.

Determining the genotype of a subject at a position of a target SNP as specified herein, e.g. as specified by the NCBI dbSNP rs identifier, may comprise directly genotyping the target SNP, e.g. by determining the identity of the nucleotide of each allele at the locus of the target SNP, and/or indirectly genotyping the target SNP, e.g. by determining the identity of each allele at one or more loci that are in linkage disequilibrium with the target SNP and which allow one to infer the identity of each allele at the locus of the target SNP with a substantial degree of confidence. In some cases, indirect genotyping may comprise determining the identity of each allele at one or more loci that are in sufficiently high linkage disequilibrium with the target SNP in question so as to allow one to infer the identity of each allele at the locus of the target SNP with a probability of at least 90%, at least 95% or at least 99% certainty.

As will be appreciated by the reader, in some cases one or more polymorphisms or alterations in linkage disequilibrium with a polymorphism or alteration disclosed herein may find use in the disclosed methods. Linkage disequilibrium (LD) is a phenomenon in genetics whereby two or more mutations or polymorphisms are in such close genetic proximity that they are co-inherited. This means that in genotyping, detection of one polymorphism as present infers the presence of the other. Thus, a polymorphism or alteration in such linkage disequilibrium acts as a surrogate marker for a polymorphism or alteration as disclosed herein. Preferably, reference herein to a polymorphism or alteration in linkage disequilibrium with another means that r²>0.7, r²>0.8, preferably r²>0.9, more preferably r²>0.95 or even r²>0.99. In particularly preferred embodiments, a SNP is considered to be in LD with a SNP set forth in Table 1 if it exhibits r²=1.0 and D′=1.0.

In one example, the SNP rs4239702 (C/T SNP) may in some cases be used, in accordance with any aspect of the present invention, as a proxy SNP for rs1883832. In particular, rs4239702-rs1883832 constitutes a GD risk haplotype C-C. Thus, presence of the risk allele C at rs1883832 may be inferred from a determination that the subject has C at rs4239702. The Applied Biosystems TAQMAN SNP Genotyping Assay context sequence for rs4239702 is provided as SEQ ID NO:13.

Linkage disequilibrium between two SNPs can be determined by any suitable method. Various software tools are available for determining LD, such as LDLink, an interactive suite of web-based tools developed to query germline variants in 1000 Genomes Project population groups of interest and generate interactive tables and plots of LD estimates, or LDLINKR, an R package designed to rapidly calculate statistics for large lists of variants and LD attributes that eliminates the time needed to perform repetitive requests from the web-based LDlink tool (Myers, T. A., et al. (2020) LDlinkR: An R Package for Rapidly Calculating Linkage Disequilibrium Statistics in Diverse Populations. Front. Genet. 11:157. doi:10.3389/fgene.2020.00157). Another available software tool is HAPLOVIEW, a comprehensive suite of tools for haplotype analysis for a wide variety of dataset sizes that generates marker quality statistics, LD information, haplotype blocks, population haplotype frequencies, and single marker association statistics (Barrett, J. C., et al. (2005) Haploview: analysis and visualization of LD and haplotype maps, Bioinformatics, 15 Jan. 2005, 21(2): 263-265, doi.org/10.1093/bioinformatics/bth457).

Haplotype determination of the subject can be performed by any suitable method. One method is to determine the haplotype of a single chromosome directly by determining the genotypes for the SNPS of the locus in a single read from one DNA molecule. Alternatively, phasing diploid genotypes of multiple SNPs into two haplotypes can be performed using any of the available software packages, such as PHASE which implements methods for estimating haplotypes from population genotype data (Stephens, M., and Donnelly, P. (2003). A comparison of Bayesian methods for haplotype reconstruction from population genotype data. American Journal of Human Genetics, 73:1162-1169), HAPCOMPASS (Aguiar D, Istrail S. HapCompass: a fast cycle basis algorithm for accurate haplotype assembly of sequence data. J Comput Biol. 2012; 19(6):577-590. doi:10.1089/cmb.2012.0084), or HAPCUT (Vikas Bansal, Vineet Bafna, HapCUT: an efficient and accurate algorithm for the haplotype assembly problem, Bioinformatics, Volume 24, Issue 16, 15 Aug. 2008, Pages i153—i159, doi.org/10.1093/bioinformatics/btn298).

As used herein, the term “risk allele” or “susceptibility allele” refers to genetic variants that are associated with an increased likelihood of an individual developing a disorder, e.g., an autoimmune disease, or associated conditions, as compared to a healthy individual.

A “therapeutically effective amount” or “effective amount”, used interchangeably herein, is that amount of a pharmaceutical agent to achieve a pharmacological effect. The term “therapeutically effective amount” includes, for example, a prophylactically effective amount. A “therapeutically effective amount” or “effective amount” of an active agent is an amount needed to achieve a desired pharmacologic effect or therapeutic improvement without undue adverse side effects. The effective amount of an active agent will be selected by those skilled in the art depending on the particular patient and the disease. It is understood that “an effective amount” or “a therapeutically effective amount” can vary from subject to subject, due to variation in metabolism of the active agent, age, weight, general condition of the subject, the condition being treated, the severity of the condition being treated, and the judgment of the prescribing physician.

In another aspect, a method for predicting response of a subject to CD40-targeted treatment comprises determining CD40 expression levels in a biological sample from a subject in need of immunosuppressant therapy; and predicting that the subject will respond to treatment with a CD40-targeted active agent when the determined CD40 expression level is greater than 0.3% of GAPDH expression level in the biological sample, or predicting that the subject is unlikely to respond to treatment with a CD40-targeted active agent when the determined CD40 expression level is below 0.3% of GAPDH expression level in the biological sample. The method can additionally comprise any of the following steps: obtaining the biological sample from the subject; administering a CD40-targeted active agent to the subject when the determined CD40 expression level is greater than 0.3% of GAPDH expression level in the biological sample; or administering a treatment that does not target CD40 to the subject when the determined CD40 expression level is below 0.3% of GAPDH expression level in the biological sample.

In certain embodiments of the method, CD40 expression levels are CD40 mRNA expression levels and are compared to GAPDH mRNA expression levels. In other embodiments of the method, CD40 expression levels are CD40 protein expression levels and are compared to GAPDH protein expression levels.

The terms “level of expression” or “expression level” in general are used interchangeably and generally refer to the amount of a polynucleotide or an amino acid product or protein in a biological sample. “Expression” generally refers to the process by which gene-encoded information is converted into the structures present and operating in the cell. Therefore, as used herein, “expression” of a gene may refer to transcription into a polynucleotide, translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a posttranslational processing of the protein, e.g., by proteolysis. “Expressed genes” include those that are transcribed into a polynucleotide as mRNA and then translated into a protein, and also those that are transcribed into RNA but not translated into a protein (for example, transfer and ribosomal RNAs).

Determination of the expression level of a gene may be performed by a variety of techniques. Generally, the expression level as determined is a relative expression level. For example, the determination comprises contacting the sample with selective reagents such as probes or ligands, and thereby detecting the presence, or measuring the amount, of nucleic acids or polypeptides of interest originally in said sample. Contacting may be performed in any suitable device, such as a plate, microtiter dish, test tube, well, glass, column, and so forth. In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the biological sample.

In a particular embodiment of the method, the expression level of CD40 gene can be determined by determining the quantity of mRNA.

Levels of mRNA, including CD40 mRNA expression levels, can be determined in a biological sample by any suitable method, including use of commercially available kits and reagents. For example, the nucleic acid contained in the samples is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Quantitative or semi-quantitative RT-PCR is preferred. Real-time quantitative or semi-quantitative RT-PCR is particularly advantageous.

Nucleic acids having at least 10 nucleotides and exhibiting sequence complementarity or homology to the mRNA of interest herein find utility as hybridization probes. It is understood that such nucleic acids need not be identical, but are typically at least about 80% identical to the homologous region of comparable size, more preferably 85% identical and even more preferably 90-95% identical. Probes typically comprise single-stranded nucleic acids of between 10 to 1000 nucleotides in length, for instance of between 10 and 800, more preferably of between 15 and 700, typically of between 20 and 500. The probes and primers are “specific” to the nucleic acids they hybridize to, i.e. they preferably hybridize under high stringency hybridization conditions (corresponding to the highest melting temperature Tm, e.g., 50% formamide, 5.times. or 6.times.SCC. SCC is a 0.15 M NaCl, 0.015 M Na-citrate).

In the context of the invention, “hybridization” relates to the fact of obtaining a close interaction of the nucleotide probe and the target region that is expected to be revealed by the detection of the nucleotide probe. Such an interaction can be achieved by the formation of hydrogen bonds between the nucleotide probe and the target sequence, which is typical of the interactions between complementary nucleotide molecules capable of base pairing. Hydrogen bonds can be found, for example, in the annealing of two complementary strands of DNA.

It will be advantageous to use nucleic acids in combination with appropriate means, such as a detectable label, for detecting hybridization. A wide variety of appropriate indicators are known in the art including, fluorescent, radioactive, enzymatic or other ligands.

Conventional methods and reagents for isolating RNA from a sample comprise High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), Oligotex kit (Qiagen), phenol extraction, phenol-chloroform extraction, TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent® (Ambion), RNAqueous Kit (Ambion), ToTALLY RNA™ Kit (Ambion), Poly(A)Purist™ Kit (Ambion) and any other methods, commercially available or not, known to the skilled person.

In one embodiment, the expression level of one or more mRNAs is determined by the quantitative polymerase chain reaction (QPCR) technique. The QPCR may be performed using chemicals and/or machines from a commercially available platform. The QPCR may be performed using QPCR machines from any commercially available platform; such as Prism, geneAmp or StepOne Real Time PCR systems (Applied Biosystems), LightCycler (Roche), RapidCycler (Idaho Technology), MasterCycler (Eppendorf), BioMark™ HD System (Fluidigm), iCycler iQ system, Chromo 4 system, CFX, MiniOpticon and Opticon systems (Bio-Rad), SmartCycler system (Cepheid), RotorGene system (Corbett Lifescience), MX3000 and MX3005 systems (Stratagene), DNA Engine Opticon system (Qiagen), Quantica qPCR systems (Techne), InSyte and Syncrom cycler system (BioGene), DT-322 (DNA Technology), Exicycler Notebook Thermal cycler, TL998 System (lanlong), Line-Gene-K systems (Bioer Technology), or any other commercially available platform. The QPCR may be performed using chemicals from any commercially available platform, such as NCode EXPRESS qPCR or EXPRESS qPCR (Invitrogen), Taqman or SYBR green qPCR systems (Applied Biosystems), Real-Time PCR reagents (Eurogentec), iTaq mix (Bio-Rad), qPCR mixes and kits (Biosense), and any other chemicals, commercially available or not, known to the skilled person. The QPCR reagents and detection system may be probe-based, or may be based on chelating a fluorescent chemical into double-stranded oligonucleotides.

The QPCR reaction may be performed in a tube; such as a single tube, a tube strip or a plate, or it may be performed in a microfluidic card in which the relevant probes and/or primers are already integrated.

In a particular embodiment, the expression level of CD40 gene may be determined by determining the quantity of protein encoded by the CD40 gene.

Such methods comprise contacting the sample with a binding partner capable of selectively interacting with the protein present in said sample. The binding partner is generally an antibody that may be polyclonal or monoclonal, preferably monoclonal.

As used herein, the term “monoclonal antibody” refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Monoclonal antibodies can be prepared by any suitable method or purchased commercially.

Alternatively, binding agents other than antibodies may be used. These may be for instance aptamers, which are a class of molecules that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. Alternative aptamers may be derived from an AFFIBODY or other peptide scaffold (Hosse review).

The binding partners of the invention such as antibodies or aptamers, may be labelled with a detectable molecule or substance, such as a fluorescent molecule, a radioactive molecule or any others labels known in the art. Labels are known in the art that generally provide (either directly or indirectly) a signal. As used herein, the term “labelled”, with regard to the antibody or aptamer, is intended to encompass direct labeling of the antibody or aptamer by coupling (i.e., physically linking) a detectable substance, such as a radioactive agent or a fluorophore (e.g. fluorescein isothiocyanate (FITC) or phycoerythrin (PE) or lndocyanine (Cy5)) to the antibody or aptamer, as well as indirect labelling of the probe or antibody by reactivity with a detectable substance. An antibody or aptamer of the invention may be labelled with a radioactive molecule by any method known in the art.

The aforementioned assays generally involve the coating of the binding partner (i.e. antibody or aptamer) in a solid support. Solid supports which can be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

In another embodiment of the invention, the measurement of CD40 in the sample may be achieved by a cytometric bead array system wherein the antibodies that bind to the biomarkers are coated directly or indirectly on beads; microbeads dyed with multiple fluorescent colors and lasers detection may be used.

For example, the level of a biomarker protein such as CD40 may be measured by using standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; Immunoelectrophoresis; immunoprecipitation.

More particularly, an ELISA method can be used, wherein the wells of a microtiter plate are coated with a set of antibodies against CD40. A sample containing or suspected of containing CD40 is then added to the coated wells. After a period of incubation sufficient to allow the formation of antibody-antigen complexes, the plate(s) can be washed to remove unbound moieties and a detectably labeled secondary binding molecule added. The secondary binding molecule is allowed to react with any captured sample marker protein, the plate washed and the presence of the secondary binding molecule detected using methods well known in the art.

The following examples are merely illustrative of the methods disclosed herein and are not intended to limit the scope hereof.

### EXAMPLES

### Example 1. CD40 Gene Polymorphisms and Prediction of Response to a CD40-Targeted Therapy

This example studies whether CD40 gene polymorphisms affect response to the CD40-targeting monoclonal antibody, iscalimab.

Blood samples were obtained from 13 patients with Graves' Disease that participated in a study of response to iscalimab in treating Graves' disease. (Kahaly et al., A Novel Anti-CD40 Monoclonal Antibody, Iscalimab, for Control of Graves Hyperthyroidism—A Proof-of-Concept Trial, The Journal of Clinical Endocrinology & Metabolism, Volume 105, Issue 3, March 2020, Pages 696-704, doi.org/10.1210/clinem/dgz013). The blood samples were blinded with respect to iscalimab response.

The blood samples were genotyped for 7 CD40 single nucleotide polymorphisms reported to be associated with Graves' Disease or other autoimmune diseases: rs6074022, rs1883832, rs745307, rs4810485, rs11569309, rs3765457, and rs112809897.

Genotyping was performed as follows: Genomic DNA was extracted and purified from whole blood using the QIAamp DNA blood kit (Qiagen, Hilden, Germany) following the manufacturer's instructions. Seven CD40 SNPs (rs1883832, rs112809897, rs3765457, rs745307, rs11569309, rs60740220, rs4810485) were genotyped using the Taqman allelic discrimination assays (Thermo-Fisher Scientific, Waltham, MA). Table 1 below tabulates the context sequence and assay for each genotyped SNP.

TABLE 1
SNP context sequences for genotyping assay

| | | | Thermo |
|---|---|---|---|
| | Applied Biosystems | | Fisher |
| | TAQMAN | ID | Scientific |
| SNP | SNP Genotyping Assay | EQ | Assay ID |
| ID | context sequence | NO | (cat #) |
| rs188 | GTCCTGCCGCCTGGTCTCACCTCGC | | C_ |
| 3832 | [C/T]ATGGTTCGTCTGCCTCTGCA | | 11655919_ |
| | GTGCG | | 20 |
| | | | (4351379) |
| rs607 | GCTGCCTGAGTGCTGAGTGTCCTCA | | C_ |
| 4022 | [C/T]GACATGGCAGACAGCTGCTT | | 29599389_ |
| | CCCCA | | 20 |
| | | | (4351379) |
| rs745 | GGAGTTGGGAGTGGGGAATGAGAAG | | C_ |
| 307 | [A/G]AAAGGGAAGGAAGACTTCGG | | 594684_ |
| | GGAAG | | 10 |
| | | | (4351379 |
| rs481 | CCTACTTTAGAGGGCTGTAGATTCC | | C_ |
| 0485 | [G/T]GCCTGAAGCCTGGGCAGGAA | | 1260190_ |
| | TGACC | | 10 |
| | | | (4351379) |
| rs115 | AACTATGGGGAGTGAGAACTGGAGA | | C_ |
| 69309 | [C/T]TGACAGACTTTTAGGGGAGC | | 31734592_ |
| | GTTTT | | 10 |
| | | | (4351379) |
| rs376 | CCTGGCACCACTGGCAGAGCCTAAC | | C_ |
| 5457 | [A/G]CTGGCTGTTCTTCACTCCTT | | 27513394_ |
| | TCCTG | | 10 |
| | | | (4351379) |
| rs112 | ATCTGAGAGTTACCCCCTCAACAAG | | C_ |
| 809897 | [C/T]TCATACCAAGCCTTGAGGAT | | 153430601_ |
| | CTGGC | | 10 |
| | | | (4351379) |

Table 2 below tabulates, for each subject, the SNP genotyping results and the subject's response to treatment with iscalimab.

TABLE 2
Genotyping results and response to iscalimab

| Patient | | | | | | |
|---|---|---|---|---|---|---|
| ID | RS6074022 | RS1883832 | RS745307 | RS4810485 | RS11569309 | RS3765457 |
| K1 | C | T | G | T | T | A |
| K2 | T | C | G | G | T | A |
| K3 | T | C | G | G | T | A |
| K4 | T | C | A | G | T | A |
| K5 | T | C | A | G | T | A |
| K6 | C | T | G | T | T | A |
| K7 | T | C | G | G | T | A |
| K8 | C | T | G | T | T | A |
| K9 | T | c | G | G | T | A |
| K10 | T | C | A | G | T | A |
| K11 | C | T | A | T | T | G |
| K12 | T | C | G | G | T | G |
| K13 | C | T | G | T | T | A |
| Disease | S¹ | D² | D³ | A & | D³ | D³ |
| | | | | SLE⁴ | | |
| Risk | | | | | | |
| Allele | | | | | | |
| | | | | | RS1883832 | |
| | | | | | Risk | |
| | | | | | Allele | |
| | | | | | (C) | Iscalimab |
| | Patient | | Haplo- | ap | Geno | Treatment |
| | ID | RS112809897 | types | Pair | type | Response |
| | K1 | C | CCGGTAC/ | | Hetero- | Non |
| | | | CTGTTAC | | zygous | Responder |
| | K2 | C | TCGGTAC | | Homo- | Responder |
| | | | | | zygous | |
| | K3 | C | TCGGTAC | | Homo- | Responder |
| | | | | | zygous | |
| | K4 | C | TCAGTAC | | Homo- | Responder |
| | | | | | zygous | |
| | K5 | T | TCAGTAC/ | | Homo- | Responder |
| | | | TCAGTAT | | zygous | |
| | K6 | C | CCGGTAC/ | | Hetero- | Non |
| | | | CTGTTAC | | zygous | Responder |
| | K7 | C | TCGGTAC | | Homo- | Responder |
| | | | | | zygous | |
| | K8 | C | CCGGTAC/ | | Hetero- | Non |
| | | | CTGTTAC | | zygous | Responder |
| | K9 | C | TCGGTAC | | Homo- | Responder |
| | | | | | zygous | |
| | K10 | C | TCAGTAC/ | | Homo- | Responder |
| | | | TCAGCAC | | zygous | |
| | K11 | C | CCAGTAC/ | * | Hetero- | Non |
| | | | CTATCGC | | zygous | Responder |
| | K12 | C | TCGGTAC/ | | Homo- | Responder |
| | | | TCGGTGC | | zygous | |
| | K13 | C | CCGGTAC/ | | Hetero- | Non |
| | | | CTGTTAC | | zygous | Responder |
| | Disease | oung | | | | |
| | | O GD | | | | |
| | Risk | | | | | |
| | Allele | | | | | |

¹Nat Genet. 2009 Jul; 41(7):824-8.
²Thyroid. 2002 Dec; 12(12):1129-35.
³Eur Thyroid J. 2013
⁴Am J Human Genetics 2000; 66: 547-556.

Although only initially tested in 13 patients, we identified two single SNPs, rs1883832 and rs4810485, which each provided 100% positive predictive value for response to therapy with CD40-targeted monoclonal antibody (Iscalimab).

The 13 patients tested could be accurately categorized as non-responders or responders to the treatment based on the rs1883832 genotype alone, with responders homozygous for C at rs1883832 and non-responders heterozygous (C/T) at rs1883832. The homozygous T allele pair at rs1883832 occurs at relatively low frequency in the general population (See Tomer et al., Thyroid 2002; 12: 1129-1135) and was not present in any of these 13 patients. However, we anticipate that testing in a larger population will demonstrate that occurrence of homozygous T at rs1883832 is also predictive of non-response to the CD40-targeted antibody.

Similarly, the 13 patients tested could be accurately categorized as non-responders or responders to the treatment based on the rs4810485 genotype alone, with responders homozygous for G at rs481048 Sand non-responders heterozygous (G/T) at rs4810485. The T allele is the minor allele at rs4810485 and the homozygous T allele pair at rs4810485 is not present in any of these patients. We anticipate that testing in a larger population will demonstrate that occurrence of homozygous T at rs4810485 is also predictive of non-response to the CD40-targeted antibody.

After genotyping the seven SNPs in the patients, we identified 3 CD40 haplotype pairs (the 5′ to 3′ ordered combination of SNP alleles on each chromosome) in the patients which we called A, B, and C (see Table 2). All patients who did not respond to iscalimab possessed haplotype pair A, comprising a rs1883832 genotype heterozygous for C, i.e. a C/T genotype, and a rs4810485 genotype heterozygous for G, i.e. a G/T genotype. Patients who responded to iscalimab possessed haplotype pair B or C, comprising a rs1883832 genotype homozygous for C and a rs4810485 genotype homozygous for G.

### Example 2. CD40 mRNA Expression Level is Predictive of Response to a CD40-Targeted Therapy

This example studies whether CD40 mRNA expression level affects response to the CD40-targeting monoclonal antibody, iscalimab.

CD40 mRNA levels were determined in the blood samples of the 13 patients described in Example 1 using the following methods. Total RNA from whole blood was isolated using QIAamp DNA blood kit (Thermo Fisher Scientific) according to the manufacturer's instructions. Total RNA was reverse transcribed using the Superscript III kit (Thermo Fisher Scientific) and Real-time RT-PCR analyses were performed in a fluorescent temperature cycler (AbiPRISM 7300; Applied Biosystems). After an initial incubation at for 2 min and 95° C. for 10 min, the reactions were cycled 40 times using the following parameters: 95° C. for 15s, 60° C. for 30s, and 72° C. for 45s. SYBR Green (Applied Biosystems) fluorescence was detected at the end of each cycle. The expression of CD40 was normalized to that of glyceraldehyde-3-phosphate dehydrogenase (GAPDH) and the relative mRNA levels were determined using the 2^{-ΔΔCt} method. The sequences for the forward and reverse primers (Integrated DNA Technologies) were, respectively, as follows:

(SEQ ID NO: 8)
5′-AAATGTCACCCTTGGACAAGCT-3′
and
(SEQ ID NO: 9)
5′-TTGTGCCTGCCTGTTGCA-3′
for CD40;
(SEQ ID NO: 10)
5′-ATGGAAATCCCATCACCATCTT-3′
and
(SEQ ID NO: 11)
5′-CGCCCCACTTGATTTTGG-3′
for GAPDH.

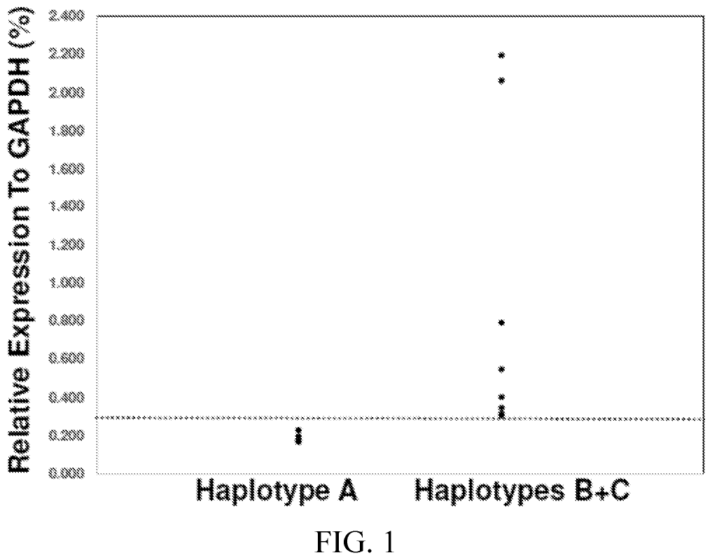

Table 3 below tabulates CD40 levels of expression compared to expression of GAPDH and iscalimab response for each of the 13 patients. FIG. 1 shows the expression levels of CD40 compared do the expression of GAPDH in patients that responded to iscalimab (carrying haplotype pairs B or C) and patients that did not respond to iscalimab (carrying haplotype pair A), and the cutoff to separate the responders from non-responders (0.3%). There was a statistically significant difference in CD40 expression levels (compared to GAPDH) in responders vs. non responders. The mean CD40 expression levels in responders were (compared to GAPDH) and in non-responders they were 0.191±0.02% (compared to GAPDH) (p=0.02, using the unpaired t-test).

TABLE 3
CD40 Expression Level and Response to Iscalimab
Treatment

| Patient | Hap. | Iscalimab Treatment | CD40 expression |
|---|---|---|---|
| ID | Pair | Response | (% of GAPDH) |
| K1 | A | Non Responder | 0.177 |
| K2 | B | Responder | 0.549 |
| K3 | B | Responder | 0.348 |
| K4 | C | Responder | 0.793 |
| K5 | C | Responder | 0.317 |
| K6 | A | Non Responder | 0.170 |
| K7 | B | Responder | 0.307 |
| K8 | A | Non Responder | 0.179 |
| K9 | B | Responder | 0.404 |
| K10 | C | Responder | 2.065 |
| K11 | A | Non Responder | 0.231 |
| K12 | B | Responder | 2.198 |
| K13 | A | Non Responder | 0.200 |

Individuals that carried haplotype pairs B or C, homozygous for C at rs1883832 and homozygous for G at rs4810485 showed significantly higher CD40 mRNA expression levels in white blood cells normalized to the expression levels of GAPDH when compared to CD40 levels of expression normalized to expression of GAPDH in individuals carrying haplotype pair A (heterozygous for C at rs1883832 and heterozygous for G at rs4810485) (p=0.02, using the unpaired t-test).

These data show that high CD40 mRNA expression levels in patients with an autoimmune disease correlated with favorable response of the patients to the CD40-targeted treatment, while low CD40 expression levels in patients with an autoimmune disease correlated with no response to the CD40-targeted treatment.

The disclosure herein include(s) at least the following aspects:

Aspect 1: A method for predicting response of a subject to a Cluster of differentiation 40 (CD40)-targeted treatment comprises genotyping Cluster of differentiation 40 (CD40) single nucleotide polymorphism (SNP) rs1883832 in a biological sample from a subject in need of immunosuppressant therapy; and predicting that the subject will respond to treatment with a CD40-targeted active agent when the determined genotype of rs1883832 is homozygous for C, or predicting that the subject will not respond to treatment with a CD40-targeted active agent when the determined genotype of rs1883832 is heterozygous or homozygous for T.

Aspect 2: The method of aspect 1, further comprising administering a CD40-targeted active agent to the subject when the determined genotype of rs1883832 is homozygous for C or when the determined genotype of rs4810485 is homozygous for G; or administering a treatment to the subject that does not target CD40 when the determined genotype of rs1883832 is heterozygous or homozygous for T or when the determined genotype of rs4810485 is heterozygous or homozygous for T.

Aspect 3: The method of any one of the preceding aspects, wherein the subject in need of immunosuppressant therapy is a patient with an autoimmune disease, a candidate for a transplant, or a recipient of a transplant.

Aspect 4: The method of any one of the preceding aspects, wherein the subject in need of immunosuppressant therapy is a patient with an autoimmune disease and wherein the autoimmune disease is Graves' disease, antibody mediated autoimmune thyroid disease, Rheumatoid arthritis, Multiple Sclerosis (MS), Myasthenia Gravis, Sjogren's syndrome, Systemic Lupus Erythematosus, Crohn's disease, or a combination thereof; preferably the autoimmune disease is Graves' disease.

Aspect 5: The method of any one of the preceding aspects, wherein the subject in need of immunosuppressant therapy is a candidate for a transplant or a recipient of a transplant, and wherein the transplant is of a tissue or an organ, preferably the transplant is of an organ and the organ is a kidney, a liver, a heart, or a lung.

Aspect 6: The method of any one of the preceding aspects, further comprising genotyping rs6074022, rs745307, rs11569309, rs3765457, rs112809897, a SNP within 1 million base pairs distance upstream or downstream from rs1883832 in strong linkage disequilibrium with rs1883832, wherein strong linkage disequilibrium is defined as coefficient of correlation (r) square (r²) value ≥0.7, or a combination thereof in the biological sample; determining a haplotype pair from the genotypes; and predicting that the subject will respond to treatment with a CD40-targeted active agent when the determined haplotype pair comprises a rs1883832 genotype homozygous for C or a rs4810485 homozygous for G, or predicting that the subject will not respond to treatment with a CD40-targeted active agent when the determined haplotype comprises a rs1883832 genotype heterozygous or homozygous for T or a rs4810485 genotype heterozygous or homozygous for T.

Aspect 7: The method of any one of the preceding aspects, wherein the CD40-targeted active agent is an antagonistic anti-CD40 antibody, preferably the anti-CD40 antibody is a fully human or a humanized antibody.

Aspect 8: The method of any one of the preceding aspects, wherein the antagonistic anti-CD40 antibody is BI 655064, ch5D12, bleselumab (ASKP 1240), Abbv-323 (ravagalimab), or iscalimab (CFZ533), preferably the anti-CD40 antibody is iscalimab.

Aspect 9: The method of any one of the preceding aspects, wherein the treatment that does not target CD40 is administration of one or more active agents in which the mode of action does not directly target CD40, surgery, and/or a behavioral or diet therapy.

Aspect 10: The method of any one of the preceding aspects, further comprising obtaining the biological sample from the subject.

Aspect 11: The method of any one of the preceding aspects, wherein the biological sample is whole blood, plasma, saliva, or a buccal swab.

Aspect 12: A method for predicting response of a subject to a Cluster of differentiation 40 (CD40)-targeted treatment, comprising determining Cluster of differentiation 40 (CD40) expression levels in a biological sample from a subject in need of immunosuppressant therapy; and predicting that the subject will respond to treatment with a CD40-targeted active agent when the determined CD40 expression level is greater than 0.3% of GAPDH expression level in the biological sample, or predicting that the subject is unlikely to respond to treatment with a CD40-targeted active agent when the determined CD40 expression level is below 0.3% of GAPDH expression level in the biological sample.

Aspect 13: The method of aspect 12, further comprising administering a CD40-targeted active agent to the subject when the determined CD40 expression level is greater than or equal to 0.3% of GAPDH expression level in the biological sample; or administering a treatment that does not target CD40 to the subject when the determined CD40 expression level is below 0.3% of GAPDH expression level in the biological sample.

Aspect 14: The method of aspect 12 or 13, wherein the subject in need of immunosuppressant therapy is a patient with an autoimmune disease, a candidate for a transplant, or a recipient of a transplant.

Aspect 15: The method of any one of aspects 12 to 14, wherein the subject in need of immunosuppressant therapy is a patient with an autoimmune disease and wherein the autoimmune disease is Graves' disease, antibody mediated autoimmune thyroid disease, Rheumatoid arthritis, Multiple Sclerosis (MS), Myasthenia Gravis, Sjogren's syndrome, Systemic Lupus Erythematosus, Crohn's disease, or a combination thereof; preferably the autoimmune disease is Graves' disease.

Aspect 16: The method of any one of aspects 12 to 14, wherein the subject in need of immunosuppressant therapy is a candidate for a transplant or a recipient of a transplant, and wherein the transplant is of a tissue or an organ, preferably the transplant is of an organ and the organ is a kidney, a liver, a heart, or a lung.

Aspect 17: The method of any one of aspects 12 to 16, wherein the CD40-targeted active agent is an antagonistic anti-CD40 antibody, preferably the anti-CD40 antibody is a fully human or a humanized antibody.

Aspect 18: The method of any one of aspects 12 to 17, wherein the antagonistic anti-CD40 antibody is BI 655064, ch5D12, bleselumab (ASKP 1240), Abbv-323 (ravagalimab), or iscalimab (CFZ533), preferably the anti-CD40 antibody is iscalimab.

Aspect 19: The method of any one of aspects 12 to 18, wherein the treatment that does not target CD40 is administration of one or more active agents in which the mode of action does not directly target CD40, surgery, and/or a diet or behavioral therapy.

Aspect 20: The method of any one of aspects 12 to 19, further comprising obtaining the biological sample from the subject.

Aspect 21: The method of any one of aspects 12 to 20, wherein the biological sample is whole blood, plasma, saliva, or a buccal swab.

Aspect 22: The method of any one of aspects 12 to 21, wherein expression levels are mRNA expression levels.

Aspect 23: The method of any one of aspects 12 to 21, wherein expression levels are protein expression levels.

In general, the invention may alternately comprise, consist of, or consist essentially of, any appropriate components herein disclosed. The invention may additionally, or alternatively, be formulated so as to be devoid, or substantially free, of any components, materials, ingredients, adjuvants or species used in the prior art compositions or that are otherwise not necessary to the achievement of the function and/or objectives of the present invention. The endpoints of all ranges directed to the same component or property are inclusive and independently combinable (e.g., ranges of “less than or equal to 25 wt %, or 5 wt % to 20 wt %,” is inclusive of the endpoints and all intermediate values of the ranges of “5 wt % to 25 wt %,” etc.). Disclosure of a narrower range or more specific group in addition to a broader range is not a disclaimer of the broader range or larger group. “Combination” is inclusive of blends, mixtures, alloys, reaction products, and the like. Furthermore, the terms “first,” “second,” and the like, herein do not denote any order, quantity, or importance, but rather are used to denote one element from another. The terms “a” and “an” and “the” herein do not denote a limitation of quantity, and are to be construed to cover both the singular and the plural, unless otherwise indicated herein or clearly contradicted by context. “Or” means “and/or.” The suffix “(s)” as used herein is intended to include both the singular and the plural of the term that it modifies, thereby including one or more of that term (e.g., the film(s) includes one or more films). Reference throughout the specification to “one embodiment”, “another embodiment”, “an embodiment”, and so forth, means that a particular element (e.g., feature, structure, and/or characteristic) described in connection with the embodiment is included in at least one embodiment described herein, and may or may not be present in other embodiments. In addition, it is to be understood that the described elements may be combined in any suitable manner in the various embodiments.

The modifier “about” used in connection with a quantity is inclusive of the stated value and has the meaning dictated by the context (e.g., includes the degree of error associated with measurement of the particular quantity). The notation “±10%” means that the indicated measurement can be from an amount that is minus 10% to an amount that is plus 10% of the stated value. The terms “front”, “back”, “bottom”, and/or “top” are used herein, unless otherwise noted, merely for convenience of description, and are not limited to any one position or spatial orientation. “Optional” or “optionally” means that the subsequently described event or circumstance can or cannot occur, and that the description includes instances where the event occurs and instances where it does not. Unless defined otherwise, technical and scientific terms used herein have the same meaning as is commonly understood by one of skill in the art to which this invention belongs.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method for predicting response of a subject to a Cluster of differentiation 40 (CD40)-targeted treatment, comprising
genotyping Cluster of differentiation 40 (CD40) single nucleotide polymorphism (SNP) rs1883832 and/or rs4810485 in a biological sample from a subject in need of immunosuppressant therapy; and
predicting that the subject will respond to treatment with a CD40-targeted active agent when the determined genotype of rs1883832 is homozygous for C or when the determined genotype of rs4810485 is homozygous for G, or
predicting that the subject will not respond to treatment with a CD40-targeted active agent when the determined genotype of rs1883832 is heterozygous or homozygous for T or when the determined genotype of rs4810485 is heterozygous or homozygous for T.

2. The method of claim 1, further comprising
administering a CD40-targeted active agent to the subject when the determined genotype of rs1883832 is homozygous for C or when the determined genotype of rs4810485 is homozygous for G; or
administering a treatment to the subject that does not target CD40 when the determined genotype of rs1883832 is heterozygous or homozygous for T or when the determined genotype of rs4810485 is heterozygous or homozygous for T.

3. The method of claim 1, wherein the subject in need of immunosuppressant therapy is a patient with an autoimmune disease, a candidate for a transplant, or a recipient of a transplant.

4. The method of claim 1, wherein the subject in need of immunosuppressant therapy is a patient with an autoimmune disease and wherein the autoimmune disease is Graves' disease, antibody mediated autoimmune thyroid disease, Rheumatoid arthritis, Multiple Sclerosis (MS), Myasthenia Gravis, Sjogren's syndrome, Systemic Lupus Erythematosus, Crohn's disease, or a combination thereof.

5. The method of claim 1, wherein the subject in need of immunosuppressant therapy is a candidate for a transplant or a recipient of a transplant, and wherein the transplant is of a tissue or an organ.

6. The method of claim 1, further comprising
genotyping rs6074022, rs745307, rs11569309, rs3765457, rs112809897, a SNP within 1 million base pairs distance upstream or downstream from rs1883832 in strong linkage disequilibrium with rs1883832, wherein strong linkage disequilibrium is defined as coefficient of correlation (r) square (r²) value ≥0.7, or a combination thereof in the biological sample;
determining a haplotype pair from the genotypes; and
predicting that the subject will respond to treatment with a CD40-targeted active agent when the determined haplotype pair comprises a rs1883832 genotype homozygous for C or a rs4810485 homozygous for G, or
predicting that the subject will not respond to treatment with a CD40-targeted active agent when the determined haplotype comprises a rs1883832 genotype heterozygous or homozygous for T or a rs4810485 genotype heterozygous or homozygous for T.

7. The method of claim 1, wherein the CD40-targeted active agent is an antagonistic anti-CD40 antibody.

8. The method of claim 7, wherein the antagonistic anti-CD40 antibody is BI 655064, ch5D12, bleselumab (ASKP 1240), Abbv-323 (ravagalimab), or iscalimab (CFZ533).

9. The method of claim 1, wherein the treatment that does not target CD40 is administration of one or more active agents in which the mode of action does not directly target CD40, surgery, and/or a behavioral or diet therapy.

10. The method of claim 1, further comprising obtaining the biological sample from the subject.

11. (canceled)

12. A method for predicting response of a subject to a Cluster of differentiation (CD40)-targeted treatment, comprising
determining Cluster of differentiation 40 (CD40) expression levels in a biological sample from a subject in need of immunosuppressant therapy; and
predicting that the subject will respond to treatment with a CD40-targeted active agent when the determined CD40 expression level is greater than 0.3% of GAPDH expression level in the biological sample, or
predicting that the subject is unlikely to respond to treatment with a CD40-targeted active agent when the determined CD40 expression level is below 0.3% of GAPDH expression level in the biological sample.

13. The method of claim 12, further comprising
administering a CD40-targeted active agent to the subject when the determined CD40 expression level is greater than or equal to 0.3% of GAPDH expression level in the biological sample; or
administering a treatment that does not target CD40 to the subject when the determined CD40 expression level is below 0.3% of GAPDH expression level in the biological sample.

14. The method of claim 12, wherein the subject in need of immunosuppressant therapy is a patient with an autoimmune disease, a candidate for a transplant, or a recipient of a transplant.

15. The method of claim 12, wherein the subject in need of immunosuppressant therapy is a patient with an autoimmune disease and wherein the autoimmune disease is Graves' disease, antibody mediated autoimmune thyroid disease, Rheumatoid arthritis, Multiple Sclerosis (MS), Myasthenia Gravis, Sjogren's syndrome, Systemic Lupus Erythematosus, Crohn's disease, or a combination thereof; preferably the autoimmune disease is Graves' disease.

16. The method of claim 12, wherein the subject in need of immunosuppressant therapy is a candidate for a transplant or a recipient of a transplant, and wherein the transplant is of a tissue or an organ, preferably the transplant is of an organ and the organ is a kidney, a liver, a heart, or a lung.

17. The method of claim 12, wherein the CD40-targeted active agent is an antagonistic anti-CD40 antibody, preferably the anti CD40 antibody is a fully human or a humanized antibody.

18. The method of claim 17, wherein the antagonistic anti-CD40 antibody is BI 655064, ch5D12, bleselumab (ASKP 1240), Abbv-323 (ravagalimab), or iscalimab (CFZ533).

19. The method of claim 12, wherein the treatment that does not target CD40 is administration of one or more active agents in which the mode of action does not directly target CD40, surgery, and/or a diet or behavioral therapy.

20. The method of claim 12, further comprising obtaining the biological sample from the subject.

21. (canceled)

22. The method of claim 12, wherein expression levels are mRNA expression levels or protein expression levels.

23. (canceled)


SEQUENCE LISTING (13 sequences)

SEQ ID NO: 1 - DNA, 51 bp, Homo sapiens
gtcctgccgcctggtctcacctcgcyatggttcgtctgcctctgcagtgcg

SEQ ID NO: 2 - DNA, 51 bp, Homo sapiens
gctgcctgagtgctgagtgtcctcaygacatggcagacagctgcttcccca

SEQ ID NO: 3 - DNA, 51 bp, Homo sapiens
ggagttgggagtggggaatgagaagraaagggaaggaagacttcggggaag

SEQ ID NO: 4 - DNA, 51 bp, Homo sapiens
cctactttagagggctgtagattcckgcctgaagcctgggcaggaatgacc

SEQ ID NO: 5 - DNA, 51 bp, Homo sapiens
aactatggggagtgagaactggagaytgacagacttttaggggagcgtttt

SEQ ID NO: 6 - DNA, 51 bp, Homo sapiens
cctggcaccactggcagagcctaacrctggctgttcttcactcctttcctg

SEQ ID NO: 7 - DNA, 51 bp, Homo sapiens
atctgagagttaccccctcaacaagytcataccaagccttgaggatctggc

SEQ ID NO: 8 - DNA, 22 bp, Artificial Sequence
  synthetic CD40 primer
aaatgtcacccttggacaagct

SEQ ID NO: 9 - DNA, 18 bp, Artificial Sequence
  synthetic CD40 primer
ttgtgcctgcctgttgca

SEQ ID NO: 10 - DNA, 22 bp, Artificial Sequence
  synthetic GAPDH primer
atggaaatcccatcaccatctt

SEQ ID NO: 11 - DNA, 18 bp, Artificial Sequence
  synthetic GAPDH primer
cgccccacttgattttgg

SEQ ID NO: 12 - DNA, 15101 bp, Homo sapiens
  gene (1744)..(13360): CD40
gttaacactggttcaagaacatcttgagggccaggtgcggtggctcatgcctgtaatctcagcactttgggaggccaaggcaggcggatcacctgaggtcaggagtttgagaccagcctggccaacgtggagaaaccccgtctctactaaaaatacaaaaattagctgggcgtggtggcgccagcctgtaatcccagctactcgggaggctgaggcaggagaattgcttgaacccaggaggcagaggttgcagtgagccgagatcatgccactgcactccagcctgggcaacagaatgagactctgtcaaaaaaaacccaaaaaccaaaaaccaaacaaacacccaaaaaacaaaacgaaacaaaaaacaaaaaaacaaaagcacgtcttcagagttcatgaacccaggaaatgtaggcacaagtgtgtgtgtttctgcaaaatgagagggtcccgagtttcctaacatacttaaagtgttttatgatgccacaaaaggctggcccactctttttttttttttgagatggagttttgcactgtcgcccaggctgaagtgcagtgatgtaatcattgcaatcatggctcactgcaaccttgacttcttgagctcaagcgatcctcctgcctcagcctcctgagtagctgggactacaggtgtttgccaccatgcctggctaacttaaaaatttctttattttgtagagatgggggtcttgccatgttgccaggctggtcttgaactcctggcctcaagcaatctccctttttggccttccaaagtgttagattacaggcgtaagccaccgcgcctggcccccacctttatttttatttttatttattatttattttttttttttttgagactgagtcttgctctgccttcgaggctggagtgcagtggcacgatctcggctcattgcaatctctgcctctgcggttcaagcgattctcctgcctcagcctcccgagtagctgggattacaggcgaacgccactacatccggttaatttttgtatttttagtagagacggagtttcactatgttggccaggttggtctcgaactcctgacctcaagtgatctgcccgcctcggcctcctaaagtgctggattacaggcgtgagccaccgcgcccggccccactcttaataaatgcctgtctccaggtgctgggtgggaggtgggatggaatggaatgaggtgaggacgcatggatgcatggatgaatggatgggaagttgagacgacgcgcccacacgagggaatttcctttgaaagagagcgaaattctgagttgggaaactcttccttgaaacgcctccccataccccagctgtggccttcccgttttctgcgtggtggtgtggggggaacttcctcaggcctctccgcagtggagcctctttcggttctgccaggatacctagaggcagcggagagcggggcagggaggggaaaaccgtgagggtccctgtggcaggccccagcacccatgggatctctctccggtcgcaggaagcaggctagctcctagcccgcctcggcttggcctttgtgggacctgggggcaaagaagaagagctgtctctgggaccatgcctcctcccgtacacagcaagatgcgtccctaaactcccgggggaattagacttgtgggaatgttctggggaaactcctgcgcggtgaattgctgggggctccgcccccccgataggtggaccgcgattggtctttgaagaccccgcccctttcctgggcggggccaaggctggggcaggggagtcagcagaggcctcgctcgggcgcccagtggtcctgccgcctggtctcacctcgctatggttcgtctgcctctgcagtgcgtcctctggggctgcttgctgaccgctgtgagttgtttttgccccgaccagacgggagttgggagtggggaatgagaaggaaagggaaggaagacttcggggaagaggccttcctggctgatttttgtgggggcaggagggtgggtgggagctgggcaaggtgcccccgctcctggctgaatggggtgggctgcctctctcttctcccgggctggggtcccgggagcggcctacaggggccgctcagggaaggcactggctgcccaagcgtgcctagacggcctggacgggtttagggagcctcagaggctggccacacagagactggtagggggttcagagggcgggaagtgaggcggaccaagggaaggggcgggtctggcccgtttcctgtccccttcttattgtggacagatgccagcctctgtaagtagttatcatctccttgccagctggggctgccttcttccagggcatcttgtgggaacaagagatgggtgcagaggcccaggtacttttgtgagaaggcaaggagcttttaacatcgccttccaccccgaaccgtatcttgggtgttccaacctaggaggaatccccagggctttgcctttttctcctgaatttaagatgacataggagacccctggggagatgaacagtttatgggacacaataaagggttaggagaccagagttctggttggctctgacagggctggtgatcagagggctggagaaaccaggggtttctccaggcaccagaggggctcagagccaaccaagcatatctccgggattttcagaagcctacacttgactcactttttgtttaaatgtatttttgtagttcctcattctggaggctgggaatcccccaagtacctggctccttcatcccagcccctctggcctccccctactttagagggctgtagattcctgcctgaagcctgggcaggaatgacccatggtatcaaggaaagcaagggaagcagcaagggaagagagggagtggggaggctgctttggtcccacagctttcactttcacctgaagcaatggctcttagggaacagggaggcagggggagggcggagctggaaagaggtaaaggggggcccttgtggtaggagtggagaaagagccagaggaggtggggtgaagggtgtgatccaggcttctcaagagcagagtttgccctcataactcccaactttggctccaggtagaggctgggctgtgacaacaatgtcagaagctatctattgagggcttcttgtgtgtcaggctctgagccaaacactgcctgttttctttgtctgatttctcacaactcccccattatacagatgggcaaattgaggctcagaaagggggattgtcttgccaaaggtctcatagctagctaatggaagaacctggttgtgaatctacatctgcatgattcccgagcctgcctctcagatagtgagagtctccaagctctggtcctgagctgttttgtggcagaaggaccagaactatggggagtgagaactggagattgacagacttttaggggagcgttttatttctcatgtgtttgaagatggtatcaaggactttcctatctttgggagtgtgggagctccacgttcacaggatggtgtcttgcaatgagctggtggggggcagtagccttttctacttcctttcccattttgggtaagacacatttctgtaagtaatttgctgagatacccaggttgaatgagagccaccagttaggtaggattctggacagccagccaggtagccgggctgcttgccatatatcatgcaagcagaaacaaatgaatgatgattaaaattgccatttaatgagcacctactatgttcctgacactgtgctaggccatatacatgtattctttcttatcttcgtaatccaacctgcagggcaggcattattactcccattttagagatagagaaactgaggctaagagaagcaaaataactagtaagtgttacaaagtcaggactggagtctaaagctgtctgactctcaaacttgtgttcttttcactggctgttcccaaactgtgggacagttttaaggagcacatggacatagaattaaacatacacttactttacagttcttttaaaaatccttctcattttttcaaagaggaagtctctggagctagaatagagttaatgcctctcaaaggcttgctaatccttcttttaaaacaaaaatcaagagcaggcctgggagggccttcaacaagcaaacaaccagctgggttttaataaccttgttttgtttccccagaatttatttttagggttaccttttatttatgagaagtgatactggttcttgtctcttggcaatgatgtgaggtttacatttaaagtaaatgtaccggccaggcacggtggcttgtgcctgtaatcccagcactttgggaggccaaggcagtcagatcacttgaggtcaggagttttagatcagcctggccaacatggtgaaaccctgtctctactaaaaatacataaattagccgggcatagtggtacacacctgtaatcccagctactcaggaggctgaggctggagaattgcttgaacccaggagatagaggttgcagtgggctgagatgatgccactgcactccagcctgggcgatggagcgagactctgtctcaaaaaataaaataaaagtattgaaattaacaataagtaattaatagcatgggtggtacctggatgtagtaaaatggtgaagatgaaacacaagttgatggagagaggagcattgagacctgagttctcatttggactctgtcactgtgagactctgggcaagtgaccctcctctttggtgctcagtctcaactatctgtaaaatgaaagtgtgagtttacccttccagctttacattctagcattttatgagggaagggctggatgaacagatgatgaggagttggaggaagaaaacatgatgggctttggaaaggagcaggaagggaagcagaagaataggaggaagaggccaagtgctaaacatagccccaaacagcactgggaccagctgaagtcagccagcttcaggactccaggggagctgctggagtccccatatcctatgggatctttgggaagaggaatgactcaggcatcaagccccaaggaattctgttctgttcagagaatattgtgagtttacagtaccattgctttgtaaaaataccagaatgattctctgggtgcgattataatcagctcagttgacaatttacttgaaaacaaacatgccaaatatcatgcaggttccactttctgttttgacttgcacttcagtttgcagcctctgtcctggatgacttttacctttctgctgaagaagttgcaacggagatttcaagatcccttcaaattgcacaattctgtttttaggtccatccagaaccacccactgcatgcagagaaaaacagtacctaataaacagtcagtgctgttctttgtgccagccaggtgagatgccaaccctctagccccatcatggagtccccctttgctttggtggcagacgcagaccccatatgttaactgtaaactcaaatctgaaacgacccatttcccagccctgcttcactgtcagaatgttctggttccctctctaccaggtaaaactctgtctaccctgaactagggatcccagcttctccatcttcctcgcctgattatgaaggatccaagactttcatctttgaatcccctaccctaaagcctggcctgatcattgtgtggttagtgtctgactcatggagttggccagagccctccctcatttcctgatgttttccaggacagaaactggtgagtgactgcacagagttcactgaaacggaatgccttccttgcggtgaaagcgaattcctagacacctggaacagagagacacactgccaccagcacaaatactgcgaccccagtgcgtgcgctgttgggaaagggacgcttgggaaccgggctgatattcccgacaatgcagccattctaattttatgtagccagggtctgctctgattggttggagtccgggctgtactgatcattaaatgatttgattgccatctctacttggaagagggtctgaggaagaaagagcaggcaatgtggggagtgaggctcagagcatggcccagcagggggttcccatccttcctgcccttctcttctcagacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattggtaagtggctcatctgggaatcagttttggagggggacagaggagcttagggcccaaggtgaggggctgggcagtgggcacttagccccagaggcagaggaagcagaggctccaacctatgtcggtatccccactggagtgagctgcagacgggaccttgttcattctgccttctgccatggggatctgcctttgaagggcaatgggagaagtcctcctggggactgcagctgtcgggggcagtaccacatcgggggaagagtgctcaaggcaggagctcttcccgtcctgcctggccactggctgccttgtgagccggacaggtggtccactgtgatggttaatgtccccctccccacccactcccagctacaggggtttctgataccatctgcgagccctgcccagtcggcttcttctccaatgtgtcatctgctttcgaaaaatgtcacccttggacaaggtataagcactcatcccttgtgtttcctgctctaagagtggcatggagctgcctccattctctccagccacctgtcctgtccctgctcccagaggtccacacacactcatgtacttgtgaagcatctgcagagtggcctcatggccaaccagacaggcacatttccacattttttttgcctgctgtctctttgaggtaatagacactgttgatctctcgcttcatgagagcctcctatcttgggggtattgggacacttattttagctttccttctgcccctcctgcttctcctcagttttcctcgtcttgctttcaccttacctggctttctagggctttctgggctctgggtgctcaccctgagggcctccctctcttacctccaactccaaacccacaccaggtcctgccactggctgtctacgtgttttgggaacttactgtctccactgttgtcactttagtttgggcctcatcactgtggtctgggtgatgccttttctgcctcctggcctccctgcctctgtctctcccctcctgctggttctgtctccatcctcttgccaacatgagcgttcgacagtttctttcaaatcatgacactctcctatttgagatgcttcctgtctctctgttggaactaagactccttagcatggcacccaaccttcctgttgcatttcctgctctctttcctgcatcgcatagcttcatgctacttgcaatcctctgaacacactgttcattctcttccatcaaactcatctgcctggaataccttaaacatgggccccaggccaggcgcggtggctcttgcctgtaatctcagcactttggatgccaaggcgggtggatcacttgaggtcaggagttcaagaccagccagcacaacatggtaaaaacccatctctactaaaaataccaaaaaattagctgggtgtggtggtgggcgcctgtaatcccagctcctcgggaggctgaggcaggagaatcacttgaacccggaaggtggagtttgcagtgagccaagatagcgccactgcactccagcctgggcaacagagcgacattctgtctcaaaaaacaaacacctgccccattaactttttgcatttgatttttaaaaatgggcaagataggcacatgggacagaaggcacaaaagagccaaagtgatgtctttctcccatccctgccccttaggctcccagttctttctggagggagccattgttccttgcatatccttccagagattctacatataaacaaaccaacacacacacacacacacacaaacacacacaaaatttccctccttttacttttgcacaaataggagtatacattttatttgttaactgtctgcctttccctaatagattgaaaattccttaaatgtagaaacttggccttttttttttcttccattgatacatcccctatacctggaacagtacctgacgcatggtaggtgcttaaatttttactgataaatgttgactgataactggaggcaccactggtatagtttttttttttttttttttttttttttttttttgagacagagtctcactctgtcgcccaggctggagtgcagtggcgcaatctcggctcactgcaagctctgcctcccaggttcacgccattctcctgcctcagcctcctgagtagctgggactataggcgcccgccaccacacccggctaatttttttgtatttttagtagagacggcgtttcaccgtgttagccaggatggtcttgatctcctgacctcgtgatccgtctgccttggcctcccaaagtgctgggattacaggcgtgagccaccgtgcccggccaccagtggtatagtattaatggaatcagtgcattggcttacgtatctgattacagctcagtaagtgtgtgaccctcactgagcctcagtctcctcatctgaaaaatgggaatgaccttcatttcacaaggcttgagctaaaaacatgtaaagtgtattgtaaattcctgaatgctctactcatgtaagactaaagtaggccgggcgtggtggctcacacctgtaattgcagcactttgggaggccgaggagggcagatcatgaggtcaagagatcgagaccatcctggctaatatggtaaaaccctgtctctactaaaaatacaaaaattagctgggcgtggtggcgcacatctgtagtcccagctactcaggaggcggaggcaggagaattgcttgaacctgggaggtggaggttgcagtgagctgagatcgcgccactgcattccagccagtctggcgaaagagcaagactctgtctcaaaaaaaaaaaaaaaaaaaaaaaaagactaaagtacatggtttcttcaaagcttctctctctttctcccaccttagatgatttttcctttgcaatgtcctgtgtccattccgccccactcctcctggggccacctggaccaggtcttcatcatctcatatctatatgtttgctgtgtctcctggctggccactcttctgtaatttctcctcctctgagctctctgggcagctgaatcttctcactagtgaagtcgcctggttggatgctgatgagactgaccagctgaatccagttgaaaacttcacacttggcagtgatctggttctaaagacacaattttccatagtttcctaacaccatcctgcatgccacctgccttatttccccacatcacatcgtcccacttagcgggactgcactgctgatccaaattttacatcctttagggcccactcaggtcatatgtcctcagggaagtctttctggaagaaccttaaaccagaggttctcaacagggggcagttttgctccctgtggaacgtttgccaatgtctggacacatttcattcgtcacaaacggagagggggatgctacagggatctggcggatagaggccagggatgctgctgaacatctgcaatgcataggacagcccacccccacccccacacccccagtaaataatgatccagcccaagtgtcactggtgctgacgttgagtaaccctatcttaagctgaactcatcatctctccattccagccttggtggattctgtctcctctgaaccattcccatctcactttagcctacctagatcacaaagcttggcactcattatagactcccctatttattactccttcaagatgtgcaagaatcttttctctgcacttttaagttctgtaagaagagtctgtgtcgttcctataataaccagcataggacgttgcacgtgttgtgtgctcagtgaacctggatttgttgattgttgactgactcactctagagttggaaatcttatgcttggggaaacttaatatctctttctttctctgtgtgtgtgcatttgtgcacgtgtctgtgcatagctgtgagaccaaagacctggttgtgcaacaggcaggcacaaacaagactgatgttgtctgtggtgagtcctggacaatgggccctggagaaagcctaggaaggtgggaactgaagggggagatgaggcacacaggaacactggatgggaaaaaggggaggggaggcagtttgggggtgtggtatcacagctctgccacttatcttgggagtctgggcaaatcacttcccctctcttagcctcagtttcttcatctgtaaaatgggatgataacagcacttccttagtaggttttgattttagagtgagaaggttggcctacagtaaagatcagataatgtaaatcagtgaaaaaggtcaggggtaagaaaattacattctctttacctaacgctaaatgaccagttaatgggtgcagcacaccaacatggtacatgtatacatatgtaacaaacctgcacattatgcacatgtaccctaaagcttaaagtataataataataaaatttaaaaaaacgaaaaatacattctctttgctttttctcaaaatgtactttcctctttgtagggctgggactagaatgaggtgagcaaggcacttgccctcgggcgcaatatttaagaaggtgccataaaagtgtagtaatcaaggtaaattcattttgatgcaatatttttaaaaataaaaattaatgcaaagaaatccatgatgagcaagatagcaacattttaaataaagaacaggatccgaccctgtgtttgcatgaccctgcctcactcacctcaccctaatcctggccctggttccagtaaaaggaataggcagccagcctgcaggccgtagtttgctgacttggtgtccgcctgatgattttcaaaatatggcattaaaagaatgtttaccttgatgactgagtgttttggacatccttttcaattttgtcctgaaacaatttcatcccttgcctcacgctagtctccgccctgccttttggtctttcttttattttcccactttgaaaaaaaaattcggcatgagaaatactttacctttcccctccactcttctataccaaaagcaacatgcagacatgaatcatgctagacctcggcattgggcagagagcagggagtggcggggagcatggtgagcaggtggtgacagccactgccaccactcgcttctagatggttcccaggtggggaggctgccaactggaacccagtcttcccagtttgtaagagaaatcagatgtctaggtttgaatatgtgatctcccagtttaaaaatgtcggcaaatatttccaaacgttaagaaaatgttctggctcctttaaagacatctgccagccacatttccccaaggaccgcggtttgaaccttctgatgtagatgagctctgacattggaagattctggagtctgacaagtcacagcaggttgagggtagggagaaactgcaggtgaggggtgcatgctgaagtcctgatttctccaggtccccaggatcggctgagagccctggtggtgatccccatcatcttcgggatcctgtttgccatcctcttggtgctggtctttatcagtgagtcctcaggtggggaggtgttgggggagggaggggagaccacctgtttcttatctggcctctccaactccccatcctttttttttttttttttttttttagaaaaggtggccaagaagccaaccaataaggtaggtcacccctgagaacccgggacagagttttgacaaactgggaagatggcctcacggttgcctatggggcagtaaaactgattcagagtctgtctctgcagccagtggggtggcagcagaattggggactgtcatccccacccaccatgctccttccatccagagctcaatcccccacagaactgcccctggcaccactggcagagcctaacactggctgttcttcactcctttcctggcattcaacgcgtggggagctgcatctttgggccttggggctgggtcaaatgggtgggagcaaatgtggcagccccttaagcccactggctcccactctggaagctcttcgtcgcccttggtgtggccagcagggggcaggaggcacccgaggaatcagcactgacccgccgtctgggaaaggggggagggcttggggaagggatccgcttcccagggaggggctcctcagaggcacagctgcccctgctgctgggggtgacctcacaccttgcctctccaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctgggggagggttggttaaagggagatttggctttcccataatgcttcatcatttttcccaaaaggagagtgaattcacataatgcttatgtaattaaaaaatcatcaaacatgtaaaaagaaaaacgggggtgaacatgctgggtgacatgagctatttaacctgctgtcaggctcacggaatgagggcattttctgtagataaataagaatgtccccaggctgctgcccctccagggtggtttccatgtgtgctcacatgtggtattgagattgcaaagtgctcttcccatttgattcatgttcacaaaaatagccttccccagcagggtgggtctgcatccctccctcttttacagaggtggaaatgaggtccagagaggcgaagtgacttgcctgtggtcacacagcggtgtctggtggtgccatgctcagagctcctcactctgggtcctgagctcctcccagatctgcctgctgtctggaactgacatctgtagctccttcccagggagatcctgggtctctcaccgccttctgacctccctgtggctgcaggagctactgcttctcggtcagatggtatccccttcccccaagcagtatctcaggataaaaataaaccatcctgttctcttttcctgctgagccaactgagggggagcctagccaggaggagcggcagatggagtgggagcagaggggaggggaggggaggaaggttggaggacaaggaggagaaggaagagcaatgcaaagtgccatgcagaaccccggcacaaaggttcgaggtgaggtttaacccaggggtctcagcctttcccaggaaagaacctgctttggatttccccaacacatcctgctggctggagggtgtggggcgatggggggcaaggggagatgtggagagggcttatcccagtggaggctgtgagggcagctgcgagccaagagaggggcacctggcttggcaggcttcccagaagagcttcaagaaccatttggatacaccaggtggaggcctttggtttagaaagtggagcaaagtggtggtggcggggaaggggaactttgggacctaattcttgtctcttatctagagcctaagagtgagtgttgttcacttctttggtggtatggtggaaagagaccaaagacccacgcctgccacagatcatctgtgtggccttggatgagtcactttcactgcttcacttctcagtttccctatctgacaaatggtgacaatatctgcagcagacgctattgatgcctcctgtgttctcttctaggtcagagcttctcaaatttgagtgtgggtcaggcgcagtggctcacgtctgtaatcccagcactctgggaggcttaggtgggcagataacctaaggtcaggagttcgagaccagcctgaccaacagggcaaaaccccgtctctactaaaaatacaaaaattagccgggcatgatggcgggtgcctgtaatcccagctacttgggaggctgaggcagaagaatcacttgaacccgggaggtggaggttgcagtgagccgagatcatgccactgtactccagcctgggcaacagagtgaaactccatctaaaataacaacaacaacaatagcaacaacaacaacaacaatagcaacaacaacaaaacttgagcgtgtatctggaccacctgcaaggtggtcctctccactattttcagaactggaaggatccttagaactcctagggacccatcatgcatccgggaacaaaagcccagaactgagtaactttcctgaggccacatagaaagctcctggccaatctctgtttagaaccagaacgtgctaacgctgccaagaataaccacctc

SEQ ID NO: 13 - DNA, 51 bp, Homo sapiens; identical to SEQ ID NO: 7